(19) United States
(12) Patent Application Publication
(10) Pub. No.: US 2002/0055185 A1
(43) Pub. Date: May 9, 2002

(54) COMPLEX CHEMICAL COMPOUND, SYNTHESIS AND VARIOUS APPLICATIONS OF SAID COMPOUND

(76) Inventors: CLAIRE MINARD, LYON (FR); CAROLE CHAIX, BRIGNAIS (FR); THIERRY DELAIR, ECHALAS (FR); BERNARD MANDRAND, VILLEURBANNE (FR)

Correspondence Address: OLIFF & BERRIDGE, PO BOX 19928, ALEXANDRIA, VA, 22320, US

(21) Appl. No.: 09/485,154

(22) PCT Filed: Aug. 3, 1998
(86) PCT No.: PCT/FR98/01731

(30) Foreign Application Priority Data
Aug. 7, 1997 (FR) 97/10300

(51) Int. Cl. G01N 33/543

(52) U.S. Cl. 436/518

(57) ABSTRACT

The invention concerns a complex chemical compound comprising a solid carrier and at least a conjugate consisting of an organic polymer and a plurality of priming biomers. The invention also concerns the synthesis of said chemical compound for biopolymer synthesis and the use of said complex chemical compound after synthesis as ligand for amplifying or detecting and/or capturing target molecules.

The present invention relates to a complex chemical compound, to a process for synthesizing it and to its use for the synthesis of biopolymers. The invention also relates to the use of the reagents obtained for, inter alia, amplifying the detection and/or capture of various biological targets.

It is nowadays known practice to use ligands, for example proteins, haptens, peptides, polypeptides, antibodies or polynucleotides, to capture target molecules or anti-ligands (biological molecules or the like), with the aim of detecting and/or assaying them, in particular in the production of diagnostic tests.

Patent application WO 91/08307 is known, which discloses reagents and a process for amplifying the capture of target molecules using oligonucleotides covalently bound to a polymer.

Patent application FR 2 707 010 is also known, which discloses reagents and a device for capturing a target molecule of sandwich type, comprising a solid support onto which is adsorbed a ligand, said ligand consisting of a conjugate resulting from the covalent coupling of an organic polymer with a plurality of biopolymers, said organic polymer being an N-vinyl-pyrrolidone copolymer.

Document EP-A-0 561 722 discloses, in one particular embodiment, a complex chemical compound which comprises:

a solid support, for example a polyvinyl chloride support,

surface chemical groups linked to the solid support via covalent bonding, these groups being known as “additional functionalizing agents”,

a conjugate comprising, on the one hand, an organic polymer, i.e. a modified maleic anhydride homopolymer or copolymer, comprising chemical side functions, which are known as “functionalizing agents”, linked to the surface groups, and chemical residues consisting of anhydride functions, and, on the other hand, biological molecules, for example proteins, linked, via non-covalent bonding, to a functionalizing agent, for example a hapten, said functionalizing agent being linked covalently to the anhydride functions of the polymer.

Patent application WO 84/03053 discloses a solid support of polysaccharide type to which is covalently bonded a synthetic polymer obtained by polymerizing comonomers, to which may be covalently bonded one or more biologically active molecules or affinity ligands such as, for example, an inhibitor, a co-factor, a prosthetic group, an enzyme, a hormone, an antibody or a nucleic acid.

Patent application FR 2 605 237 discloses a porous support, for example silica, to which is bound by adsorption a polymer derived from polyvinylimidazole which comprises SH functions, and its use for purifying or separating proteins.

Patent application EP 591 807 discloses a polymer onto which is covalently grafted one or more biologically active molecules of the same nature. These molecules can be, for example biotin, digoxin, digitoxigenin or oligonucleotides comprising from 1 to 80, preferably 15 to 50 and in particular 20 to 35 nucleotide units.

Patent application FR 2 019 083 discloses a water-soluble polymer-enzyme binary system, used for treating substrates enzymatically, such that after treatment, the products of the enzymatic reaction are separated from the soluble enzyme-polymer product by passing them through a semi-permeable membrane.

It is thus clear that these polymers grafted with several oligonucleotides are of great interest for amplification in detection tests.

Until now, oligonucleotide ligands/organic polymers have been obtained by chemical grafting, covalently, of said oligonucleotides presynthesized on the organic polymer which is reactive or has been made reactive.

This chemical grafting is a difficult step to carry out in that it does not provide an optimum orientation of the oligonucleotides and in that it can lead to aggregated compounds whose oligonucleotide accessibility is limited (Syntheses and Characterisation of Conjugates of Nucleic Acid Probes and 6-Aminoglucose-based Polymers, Polymers for Advanced Technologies, (1996) volume 8, pp. 297-304).

A subject of the present invention is thus a complex chemical compound which can simplify and improve the production of the ligands according to the prior art, while at the same time conserving them.

According to the present invention, starting biomonomers, which are optionally chemically modified, are linked covalently, on the one hand to the chemical residues of said organic polymer, respectively, and on the other hand each to a biopolymer.

The complex chemical compound according to the invention, which can constitute an intermediate reagent, allows ligands to be synthesized directly, these ligands themselves being linked via a bond, which can optionally be cleaved, to a solid support.

Cleavage of the organic polymer/solid support bond restores to the natural state the synthesized biopolymers bound to the organic polymer in the form of ligands. The bond between the starting biomonomers and the organic polymer cannot be cleaved under the conditions for cleaving the organic polymer/solid support bond.

The advantages of the present invention are numerous since it is possible, after synthesizing the biopolymers, either to use the ligand without further modification, or to cleave the solid support/organic polymer covalent bonds in order to use the ligands. In the case in which the covalent bonds are not cleaved, this ligand can be used for amplifying the detection and capture of target molecules or for other uses. The advantage of this type of ligand is that the biopolymers of the ligand are correctly oriented, and do not have any aggregates. In the case in which the covalent bond is cleaved, ligands are obtained which can be used for amplifying the detection and capture of target molecules, said ligands being correctly oriented, and not having any aggregates. Furthermore, these various ligands can have identical or different biopolymers, at least of mixed nature, such as oligonucleotides/peptides.

The expression “solid support” means any material which is relatively inert in the native state, which can be functionalized, to which can be bound a reactive organic polymer as defined below, and which can be used as a support in detection tests, in affinity chromatography and in separation processes. Chemically modified or unmodified, natural or synthetic materials can be used as solid support, in particular polysaccharides such as cellulose-based materials, for example paper, cellulose derivatives such as cellulose acetate and nitrocellulose, dextran, polymers such as polyvinyl chloride, polyethylene, polystyrene or polyamide, or copolymers based on vinyl and aromatic monomers, unsaturated carboxylic acid esters, vinylidene chloride, dienes or compounds containing nitrile functions (acrylonitrile), vinyl chloride/propylene or vinyl chloride/vinyl acetate copolymers, copolymers based on glycidyl methacrylate and on ethylene dimethyl methacrylate, copolymers based on styrene or on substituted styrene derivatives, natural fibers such as cotton and synthetic fibers such as a polyamide, inorganic materials such as silica, glass, ceramics or quartz, lattices, i.e. colloidal aqueous dispersions of water-insoluble polymer, magnetic particles and metal derivatives.

In one preferred embodiment according to the invention, the complex chemical compound comprises, as functional solid support, a support of inorganic or organic type, more preferably activated silica or functionalized polystyrene.

The solid support can be, without limitation, in the form of a microtitration plate, a sheet, a cone, a tube, a well, beads, particles or the like.

The term “functional” means the characteristic by which the support has been made chemically reactive by any so-called “functionalization” method which is conventional or known per se to those skilled in the art, depending on the chemical nature of the support, and which generates said surface groups.

The term “ligand” means a complex formed from a reactive organic polymer coupled to a plurality of biopolymers, said ligand being, for example, the complex chemical compound after synthesis of the biopolymers, with or without cleavage of the solid support/organic polymer bond, said ligand being capable of binding to anti-ligands.

In the present invention the term “conjugate” means an organic polymer linked to a plurality of starting biomonomers.

The term “biopolymer” means any molecule which can be synthesized in an automatic synthesizer, such as enzymes, hormones, receptors, antigen determinants, antibodies, DNA, RNA, peptides, glycopeptides, oligosaccharides, derivatives thereof and synthetic analogs.

The term “biomonomer” means any base unit whose polymerization by addition of synthons leads to a biopolymer as defined above; in particular biomonomers which have been chemically modified in order to bind to the organic polymer and to act as a primer for the polymerization of the biopolymer. Mention may be made of amino acids, nucleosides, nucleotides and saccharides, and derivatives or analogs thereof.

The term “synthon” means any biomonomer which allows the synthesis of biopolymers.

The expression “cleavable covalent bond” means any chemically fixed bond which can be cleaved by a chemical, photochemical, thermal or enzymatic reaction.

In one preferred embodiment according to the invention, the starting biomonomers, which may be identical or different, are of nucleotide and/or peptide and/or saccharide type. More preferably, the biomonomers are of nucleotide and peptide type.

The expression “reactive organic polymer” means any natural or synthetic polymer or copolymer which has a linear or branched, random, alternating, grafted or block, essentially carbon-based backbone bearing, once activated, substituents for carrying out covalent reactions with the solid supports and the starting biomonomers. The polymer is preferably a copolymer. It bears biopolymers as side substituents linked directly or indirectly to the polymer backbone via covalent bonds, by means of chemical side residues. It bears other side substituents, which may be identical to or different from the previous ones, as side substituents linked directly or indirectly to the solid support via cleavable covalent bonds, by means of chemical side functions.

The copolymer units which are not involved in establishing a covalent bond with the starting biomonomers or the solid support are used in particular to space out, in the copolymer, the units bearing the starting biomonomers, and can thus serve to modify, in a known manner, the properties of the copolymer, for example the solubility properties.

Preferably, the polymer is a polymer bearing reactive functions of electrophilic and/or thiol and/or disulfide type.

More preferably, the polymer is a linear copolymer of maleic anhydride, such as poly(maleic anhydride-alt-methyl vinyl ether), poly(maleic anhydride-alt-ethylene), poly(maleic anhydride-alt-styrene) and poly(maleic anhydride-alt-N-vinylpyrrolidone) and can also be a copolymer of (N-vinylpyrrolidone/N-acryloxysuccinimide).

In one preferred embodiment according to the invention, the reactive organic polymer or copolymer according to the invention has a molecular mass of between 10,000 and 1,000,000, more preferably between 30,000 and 70,000.

The copolymer comes, for example, from the copolymerization of a maleic anhydride monomer and from a second suitable monomer, for example methyl vinyl ether, to allow the establishment of covalent coupling between the copolymer and the solid support, on the one hand, and the copolymer and the starting biomonomers, on the other hand. The maleic anhydride monomer bears carbonyl substituents, which can react with a hydroxyl function of the solid support to form a covalent bond of ester type which can be cleaved under predetermined basic conditions, and for others can react with a primary amine function of a starting biomonomer to form a covalent bond of amide type which cannot be cleaved under the predetermined basic conditions.

By way of example, the reactive organic polymer is a linear copolymer of maleic anhydride-alt-methyl vinyl ether.

When the solid support is itself a polymer or a copolymer, it should be understood that it is, in this case, different from the reactive polymer, for example in its chemical nature.

The expression “reactive organic polymer” refers to the fact that the polymer or copolymer bears, before or after activation, reactive chemical substances, in particular of electrophilic and/or thiol and/or disulfide type. These substituents can be, for example, aldehyde, epoxy, haloalkyl, ester, carbonyl, isocyanate, isothiocyanate, activated carbon-carbon double bond, maleimide and vinyl sulfone groups.

In general, the expressions “side function”, “surface group” and “side residue” mean a reactive chemical function which makes it possible to form any covalent bond; it relates, for example, to the electrophilic and/or thiol and/or disulfide radicals mentioned above, or to any group known to those skilled in the art, and is chosen as a function of the desired covalent bond.

The terms “target”, “target molecule” and “anti-ligand” mean any molecule which can be linked to the ligands via biopolymers, in particular nucleic acids such as DNA or RNA or fragments thereof, which may be single-stranded or double-stranded, antigens, haptens, peptides, proteins, glycoproteins, hormones, antibodies, oligosaccharides, medicinal products, derivatives thereof and synthetic analogs.

The ligand/anti-ligand reaction can take place directly or indirectly.

In a reaction of direct type, the ligand is specific for the target molecule. The ligand is chosen in particular so as to be capable of forming a ligand/target molecule duplex. By way of example, the duplex can be represented in particular by any antigen/antibody, antibody/hapten, chelating agent/chelated molecule couple, polynucleotide/polynucleotide, polynucleotide/nucleic acid, oligosaccharide/oligosaccharide and hormone/receptor hybrid.

In a reaction of indirect type, the ligand is capable of forming a duplex with a difunctional reagent comprising an “anti-ligand” group, responsible for forming the duplex with the ligand, and in which said anti-ligand group is bonded, in particular covalently, in a known manner, to a partner group of the target. The partner group of the target is a group capable of binding with the target (forming a target/partner complex) and is thus capable of capturing the target, under the test conditions, by establishing a bond which is sufficiently strong to ensure target/partner interaction, for example by covalent bonding and/or by ionic interaction and/or by hydrogen bonding and/or by hydrophobic-hydrophilic bonding. In this case, the ligand/anti-ligand duplex can be any couple mentioned above for the reaction of direct type, or alternatively a biotin/streptavidin or lectin/sugar duplex or the like. In particular, the ligand/anti-ligand complex is a polynucleotide/polynucleotide hybrid. The partner/target complex is of the same type as the ligand/target complex mentioned above for the reaction of direct type.

A second subject of the invention is the process for the chemical synthesis of a complex chemical compound which is the subject of the invention and as described above, comprising a plurality of biomonomers, this process comprising the following steps:

a) a functional support comprising surface groups is provided;

b) at least one reactive organic polymer is provided, the backbone of which comprises, on the one hand, chemical side functions complementary to the surface groups of the solid support, and, on the other hand, chemical side residues, said solid support being inert with respect to said organic polymer;

c) starting biornonomers, which may be identical or different, for biopolymerization are provided, comprising, on the one hand, a reactive substituent, and, on the other hand, a protective group;

d) at least one organic polymer is reacted:

either with the solid support, to establish at least one covalent bond between a surface group of said solid support and a side residue of said organic polymer, after which the organic polymer linked to the solid support is reacted with a plurality of starting biomonomers, to graft these biomonomers covalently, directly or indirectly, with a plurality of side residues of said organic polymer:

or with a plurality of starting biomonomers, to graft these biomonomers covalently, directly or indirectly, with a plurality of side residues of said organic polymer, after which the organic polymer linked to the starting biomonomers is reacted with the solid support, to establish at least one covalent bond between a surface group of said solid support and a residue of said organic polymer.

The above synthetic process is preferably carried out by reacting at least one organic polymer with a plurality of starting biomonomers, to graft these biomonomers covalently, directly or indirectly, with a plurality of side residues of said organic polymer, after which the organic polymer linked to the starting biomonomers is reacted with the solid support, to establish at least one covalent bond between a surface group of said solid support and a residue of said organic polymer.

According to yet another embodiment of the invention, the process comprises, after step c and before step d, the step:

c′) the organic polymer is reacted with a reagent which generates spacer arms, to graft a plurality of spacer arms onto a plurality of side residues of said organic polymer, respectively, each spacer arm comprising a reactive function at its free end.

For example, the spacer arm can be:

R—O—(CH₂)ₙ—NH₂

in which R is a dimethoxytrityl or tert-butyldimethylsilyl group or a photolabile group.

According to another preferred embodiment, the chemical compound according to the invention is characterized in that the starting biomonomers are extended and polymerized with synthons, each according to a predetermined sequence in order to obtain biopolymers.

According to one very preferred embodiment of the invention, the chemical compound according to the invention is characterized in that the biopolymers are of oligonucleotide and/or peptide and/or oligonucleotide-peptide type.

According to another very preferred embodiment of the invention, the chemical compound is characterized in that the biopolymers form, with the organic polymer, ligands capable of binding directly or indirectly to anti-ligands.

A third subject of the invention is the use of the complex chemical compound according to the invention to carry out the synthesis of biopolymers, comprising the steps in which:

f) identical or different synthons are provided,

g) the biopolymer chains are grown from starting biomonomers, respectively, by successive cycles of coupling/deprotection, according to at least one same predetermined sequence of synthons.

In order to carry out syntheses of different biopolymers, whether these are oligonucleotides/oligonucleotides or peptides/peptides or oligonucleotides/peptides, a person skilled in the art can select suitable starting biomonomers with protecting groups that are sensitive or insensitive to the chemical reactions carried out. Thus, it is possible to carry out a first synthesis of a first group of biopolymers while at the same time protecting a second group of protected starting biomonomers, and then to carry out a second synthesis of biopolymers on the second group of deprotected starting biomonomers.

There are many advantages to synthesizing biopolymers on the complex chemical compound according to the invention. This direct synthesis on the reactive polymer linked to the solid support makes it possible to obtain a well-defined biopolymer which is correctly oriented for detecting and/or capturing target molecules of identical nature. Furthermore, the synthesis makes it possible to obtain different biopolymers, such as oligonucleotides and peptides, on the same reactive polymer. It is also possible, by this facilitated synthesis, to obtain dibiopolymers such as oligonucleotides-peptides in several steps, which can make it possible, for example, to detect nucleic acid and protein material in the same sample.

A fourth subject of the invention is the complex chemical compound obtained by the process described above and its use for various applications, in particular for amplifying the capture and/or detection of biological targets, under various formats of bioassays (microtitration plate, chromatography, bands, etc.), oligonucleotide sequencing, directed mutagenesis, in therapy and other applications suited to the use of the complex chemical compound according to the invention.

Insofar as the covalent bonds linking the support to the organic polymers is not cleaved, the reagent obtained can be used to amplify the capture and detection of biological targets.

In one preferred embodiment according to the invention, a complex chemical compound is synthesized, this compound comprising:

a functional solid support comprising chemical surface groups,

at least one conjugate comprising:

a reactive organic polymer whose backbone comprises, on the one hand, chemical side functions that are reactive with respect to the surface groups of the solid support, the functions being linked to these groups by covalent bonding, and on the other hand, chemical side residues,

a plurality of branched mixed starting biomonomers on the organic polymer, respectively linked to said chemical residues of said organic polymer via a covalent bond, characterized in that the mixed starting biomonomers are extended and polymerized with other synthons, each according to a predetermined sequence of said synthons, in order to obtain mixed biopolymers, such as oligonucleotide and peptide.

In another preferred embodiment according to the invention, the chemical compound comprises biopolymers of oligonucleotide and peptide or oligonucleotide-peptide type.

In yet another embodiment according to the invention, the complex chemical compound comprises biopolymers forming, with the organic polymer to which they are linked, ligands which can be linked directly or indirectly to anti-ligands.

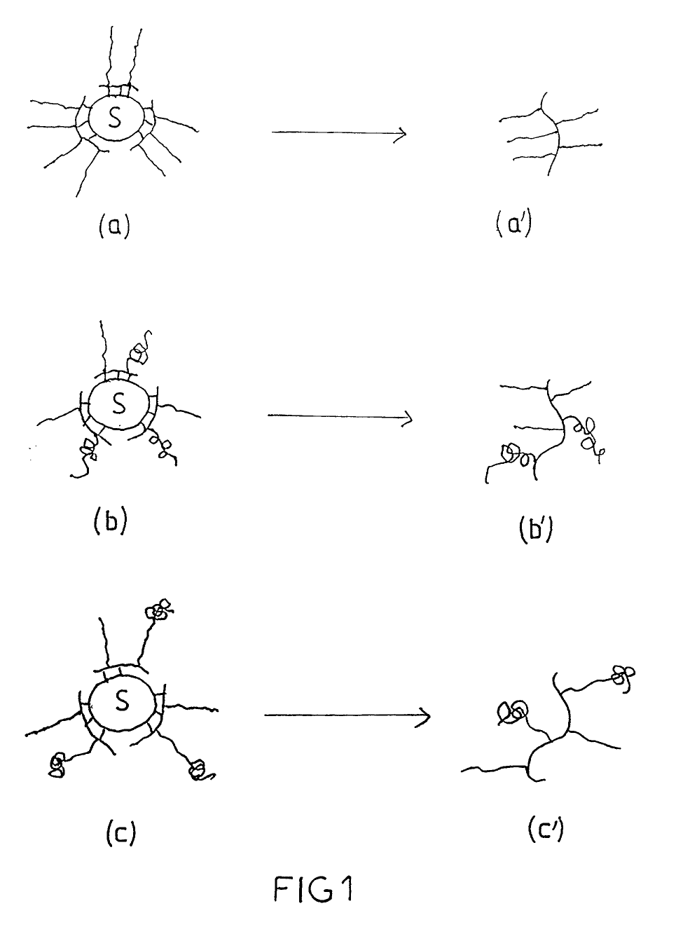

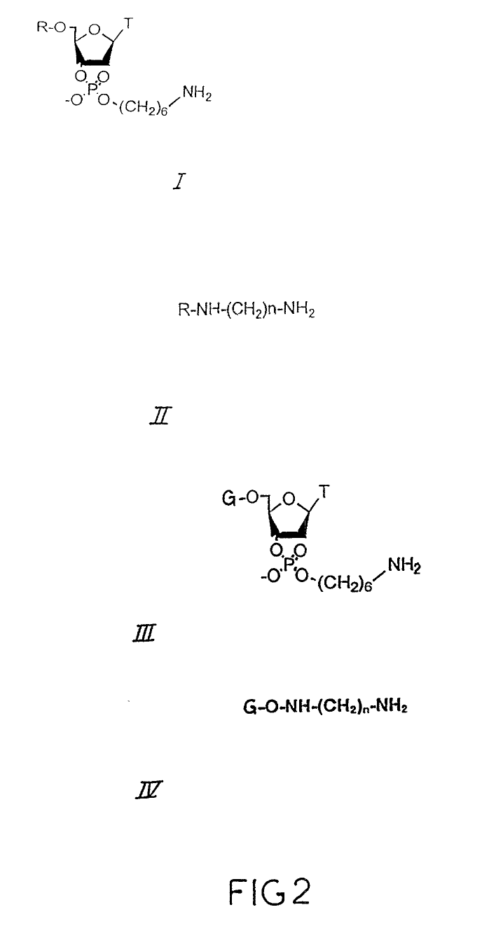

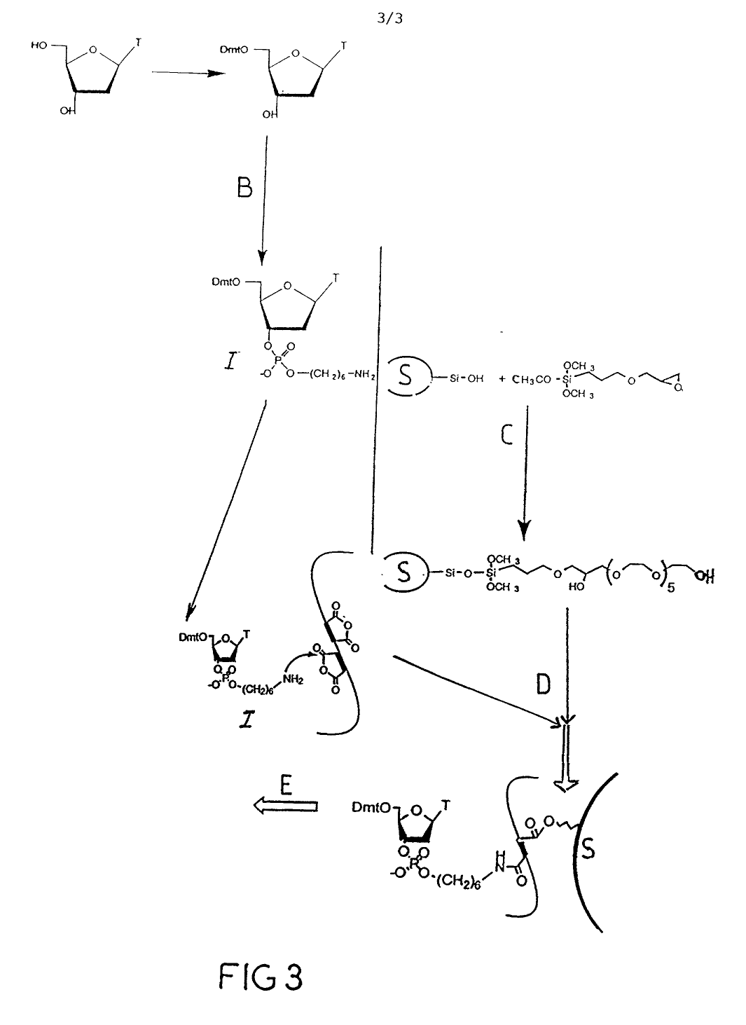

Insofar as the covalent bonds linking the support to the organic polymers are cleaved, reagents or ligands, in particular ligands, are obtained consisting of an organic polymer linked to biopolymers of mixed nature, for example peptides/oligonucleotides, which can be used for amplifying the capture and detection of target molecules These polymer/oligonucleotide/peptide complex compounds can also be used therapeutically. Specifically, insofar as the oligonucleotide can be an antisense agent and the peptide can be fusogenic, the compound can be used to regulate gene expression and to optimize the internalization of the complex into the cells. In FIGS. 1 and 3, “S” means “support”. In FIG. 2, “G” means “photolabile group”.

The figures attached are given for the purpose of illustration and do not in any way limit the scope of protection of the present invention.

FIG. 1 represents various possible forms of the complex chemical compound according to the invention, and of the corresponding ligand.

FIGS. 1(a) and 1(a′) represent the complex chemical compound and the corresponding ligand, the biopolymers of which are oligonucleotides with the same nucleic acid sequence.

FIGS. 1(b) and 1(b′) represent the complex chemical compound and the corresponding ligand, the biopolymers of which are oligonucleotides and peptides.

FIGS. 1(c) and 1(c′) represent the complex chemical compound and the corresponding ligand, the biopolymers of which are oligonucleotides and oligonucleotide-peptides.

FIG. 2 represents four examples of oligomeric primers.

The radicals R are:

for the monomer I: dimethoxytrityl, tertbutyldimethylsilyl, photolabile group,

for the monomer II: monomethoxytrityl, fluorenylmethoxycarbonyl, t-butoxycarbonyl, photolabile group.

FIG. 3 represents the general scheme for synthesizing a complex chemical compound according to Example 1.

### EXAMPLE 1

Synthesis of oligonucleotides (HBV Capture 26 mer sequence and other sequences which can be used for medical diagnosis) on a CPG 2000 A porous support coated with the linear copolymer P(MAMVE) (maleic anhydride-alt-methyl vinyl ether) (number-average molecular mass (Mn) 67,000).

A) Materials and Methods

Fluka silica beads (CPG, controlled pore size glass) of diameter 2000 Å, with a particle size of 40-85 μm and a surface area of 9.2 m²/g are used. The glass beads (100-150 μm) are obtained from the company Polysciences Inc.

The ¹H-NMR spectra were recorded on a Bruker AM400 spectrometer operating in Fourier transform mode. The ¹H chemical shifts were expressed in ppm with reference to TMS. Assignment of the ¹H signals was made by homonuclear ratio ¹H-¹H two-dimensional NMR experiments. The mass analyses were performed using an FAB+ ZAB2-SEQ spectrometer with a thioglycerol matrix.

The polymer used is an alternating copolymer of maleic anhydride and methyl vinyl ether P(MAMVE) supplied by Polysciences Inc. (Mn 67,000 g/mol).

The oligodeoxyribonucleotide syntheses were accomplished on an ABI 394 machine (Applied Biosystems, San Francisco, USA) using the chemistry-standard of DNA of the cyanoethyl N,N-diisopropylaminophosphoramidite type.

SEC-MALLS experiments were carried out in line with the following size-exclusion high performance chromatography device. Two combined columns (Waters Ultra-Hydrogel 500 and 1000 or Waters Ultra-Hydrogel 1000 and 2000) and a Waters 510 high performance liquid chromatography pump run with a buffer based on boric acid of pH 10 as eluent at a flow rate of 0.5 ml.min−1. For the detection part, a Waters 484 absorbance detector, a Waters 410 differential refractometer and a Mini Dawn F three-angle detector (Wyatt Technology) functioning at 690 nm were simultaneously used.

B) Synthesis of 3′-amino-modified dT (5′ -dimethoxytrityl-2′-deoxythymidine 3′-(6-aminohexyl)phosphate (I) (Starting Biomonomer for Nucleotide Polymerization)

200 mg (0.37 mmol) of 5′-dimethoxytrityl-2′-deoxythymidine were dried by co-evaporations based on anhydrous pyridine and anhydrous acetonitrile, and the dry material was then dissolved in 7.5 ml of acetonitrile. 1H-Tetrazole (0.55 mmol of a 0.35M acetonitrile solution) and 6-(trifluoroacetylamino)hexyl 2-cyanoethyl N,N-diisopropyl phosphoramidite (0.44 mmol of a 0.3M acetonitrile solution) were added slowly through the septum and the solution was stirred at room temperature. After one hour, 4 ml of an iodine solution (100 mM iodine in 2% H₂O, 20% pyridine, 75% THF) were added dropwise over 5 min and the mixture was stirred for a further 10 min and then concentrated under reduced pressure. The residue was dissolved in CH₂Cl₂ and the organic mixture was washed once with 5% NaHCO₃, twice with water, dried over Na₂SO₄ and evaporated to dryness under vacuum. The residue was then resuspended in a solution of 700 l of ethanol and 6 ml of aqueous 30% NH₄OH and the deprotection reaction was carried out for 16 h at 60° C., in a sealed flask. After concentration under vacuum, the residue was subjected to chromatography on a column of silica gel in CH₂Cl₂/5% TEA, and elution with a methanol gradient, to give compound (I) (46% yield). ¹H NMR (DMSO-d6): 1.32 (m, 2H, C—CH2-C), 1.37-1.42 (m, 4H, PO—CH2-CH2-CH2-C), 1.55 (t, 2H, C—CH2-CH2-NH2), 2.21-2.34 (m, 2H, H2′H2″), 2.71 (t, 3H, C—CH2-NH2), 3.16-3.24 (m, 2H, H5′H5″), 3.57 (t, 2H, PO—CH2-C), 3.71 (s, 6H, O—CH3), 4.07 (s, 1H, H4′), 4.-((S, 1H, H3′), 6.16 (t, 1H, H1′), 7.21-7.36 (m, 13H, Har), 7.46 (s, 1H, H6), 7.8-8.6 (large peak, 2H, NH2). On mass spectrometry, the exact mass given by the analysis is 724.3009 g/mol, which corresponds to the calculated mass (724.2999 g/mol).

C) Functionalization of CPGs or of Glass Beads (Solid Support)

The functionalization step was adapted from a previous procedure published by Southern et al. (Maskos, U, and Southern, E. M. (1992) Nucleic Acids Res., 20, 1679-1684). 3 g of an unmodified CPG and 5 g of glass beads were suspended in 6 ml of a sulfuric chromic acid solution (solution saturated with chromium (VI) oxide in 95% sulfuric acid) (Prolabo). After activation for 3 h at 110° C., most of the surface silane groups were in the form of silanol groups. The supports were filtered off and washed carefully with water. After washing quickly with dry acetone, the supports were dried under vacuum for half an hour and were immediately used for the silanization.

The activated beads were suspended in 28 ml of a solution (6.1 ml of 3-glucidoxypropyltrimethoxy-silane, 1.7 ml of triethylamine, 20.2 ml of dry toluene). The supports were stirred gently overnight at 90° C. and were then washed carefully with anhydrous acetone and dried for 3 h at 110° C.

In a second step, the beads were suspended in 10 ml of hexaethylene glycol with 6 l of sulfuric acid (at a concentration of more than 95%). The mixture was stirred gently and the reaction was then carried out overnight at 90° C., after which the beads were washed with anhydrous acetone and dried in a desiccator.

D) General Procedure for Grafting the Conjugate P(MAMVE)-nucleotide (I) onto Silica Beads

The starting nucleotide for nucleotide polymerization according to B) was coupled with the P(MAMVE) copolymers according to the reaction principle described in FIG. 3.

15 mg (224 mmol) of copolymer were dissolved in 1 ml of anhydrous dimethyl sulfoxide (DMSO) at 37° C. In parallel, 10 mg (13.8 mol) of (I) were dissolved in 1 ml of the same solvent. 20 nmol of copolymer, 2 mol of nucleotide (I) and 1 mal of 4-dimethylaminopyridine (DMAP) were successively added to an appropriate volume of DMSO to make a final volume of 1 ml for the reaction. After stirring at room temperature for 1 hour, 90 mg of silica beads were added to the mixture and the grafting reaction was allowed to continue overnight. The supports were then filtered off and washed gently with DMSO and anhydrous acetone, and then dried under vacuum for several hours. The amount of surface dimethoxytrityl was estimated by an acidic treatment of the silica.

E) Oligonucleotide Synthesis

The sequence chosen is an HBV (hepatitis B virus) capture sequence.

5′-TCA ATC TCG GGA ATC TCA ATG TTA GT-3′

0.1 mol or 0.15 mol of functionalized support was used for the synthesis, using a standard protocol of a 0.2 mol coupling cycle. Before the synthesis, various blocking times were investigated for the residual hydroxyl functions of the solid support (capping). During the synthesis, the yield for the coupling reaction steps was determined automatically by measuring the amount of cations of dimethoxytrityl type released at each coupling/deprotection step. Various basic treatments were applied for the cleavage and deprotection steps. After desalinating (in the case of the deprotection with NaOH) and concentration under vacuum, the molar mass distributions of the conjugates (copolymer-ODN) were analyzed by SEC-MALLS. Yields of 98% per cycle were obtained.

### EXAMPLE 2

The same synthetic principle as in Example 1 is employed, using other maleic anhydride copolymers as reactive organic polymer, such as (maleic anhydridealt-ethylene), (maleic anhydride-alt-styrene), (maleic anhydride-alt-N-vinylpyrrolidone) or P(MAMVE) copolymers of another size (MM 10,000 to 1,000,000) 224 nmol of polymer were dissolved in 1 ml of anhydrous DMSO at 37° C. In parallel, 10 mg (13.8 mol) of the nucleotide (I) modified at the 3′ end with an amino arm, were dissolved in 1 ml of DMSO. 20 nmol of copolymer, 2 mol of (I) and 1 mol of dimethylaminopyridine (DMAP—solution at a concentration of 10 mg/ml in DMSO) were dissolved in a sufficient amount of anhydrous DMSO to give a final volume of 1 ml. The reaction was stirred at room temperature for 1 hour after which 90 mg of CPG 2000 A silica beads (Control Pores Glass) or 100 mg of glass beads functionalized with a hydroxyl spacer were added to the solution. The reaction was continued overnight at room temperature. The supports were then filtered off and washed thoroughly with DMSO and anhydrous acetone, and then dried under vacuum in the presence of calcium chloride.

These supports were then used to carry out the oligonucleotide syntheses as described in Example 1, a capping step of 11 minutes being necessary beforehand.

### EXAMPLE 3

Same synthesis as in Example 2, using the NVP-NAS (N-vinylpyrrolidone/N-acryloxysuccinimide) linear copolymer as reactive organic polymer instead of P(MAMVE)

### EXAMPLE 4

Same synthetic principle as in Example 1, using a polystyrene-based solid support.

224 nmol of polymer were dissolved in 1 ml of anhydrous DMSO at 37° C. In parallel, 10 mg (13.8 mol) of the nucleotide (I), modified at the 3′ end with an amino arm, were dissolved in 1 ml of DMSO. 20 nmol of copolymer, 2 mol of (I) and 1 mol of dimethylaminopyridine (DMAP—solution at a concentration of 10 mg/ml in DMSO) were dissolved in a sufficient amount of anhydrous DMSO to give a final volume of 1 ml. The reaction was stirred at room temperature for 1 hour, after which 90 mg of polystyrene-based particles (resin of co-polystyrene-1% divinylbenzene, conventionally known as Wang resin) functionalized with 4-hydroxymethylphenoxymethyl ends, or 90 mg of polystyrene-co-divinylbenzene (solid support) functionalized with 4-hydroxymethyl-phenylacetamidomethyl ends (PAM resin), or 90 mg of a composite support based on polyacrylamide and on an inorganic matrix (Kieselguhr) functionalized with 4-hydroxymethyl-phenoxyacetyl ends (Novabiochem), or 90 mg of a polystyrene support functionalized with polyethylene glycol ending with a hydroxyl or poxybenzyl alcohol function (Novabiochem), or 90 mg of any polystyrene-based resin functionalized with a hydroxyl spacer arm, were added to the solution. The reaction was stirred overnight at room temperature. The supports were then filtered off and washed thoroughly with DMSO and anhydrous acetone, and then dried under vacuum in the presence of calcium chloride.

These supports were then used to carry out oligonucleotide syntheses as described in Example 1, a capping step of 11 minutes being necessary beforehand.

### EXAMPLE 5

The polystyrene-based supports are functionalized as described above with P(MAMVE) and the biomonomer (I) and/or the monomer (II) with the aim of starting a peptide synthesis on a solid phase using Fmoc chemistry.

In the case of the mixed oligonucleotide/peptide syntheses, the nucleic acid fragments are synthesized before the peptides.

### EXAMPLE 6

Oligonucleotide or peptide syntheses are carried out on a spherical support based on porous or nonporous silica, or on polystyrene functionalized with a maleic anhydride linear copolymer linked to a support via a spacer arm which is stable in basic medium. The aim is to not detach the conjugate (copolymer/biomolecules) after synthesis These reagents may be used for capturing biological species (gene fragments, antigen, antibody) directly from biological media.

The procedure for synthesizing the support is the same as that described in Example 2 and in Example 5, but an aminated and not hydroxylated solid support is used.

224 nmol of polymer were dissolved in 1 ml of anhydrous DMSO at 37° C. In parallel, 10 mg (13.B mol) of nucleotide (I) modified at the 3′ end with an amino arm, were dissolved in 1 ml of DMSO. 20 nmol of polymer, 2 mol of (I) and 1 mol of dimethylamino-pyridine (DMAP—solution at a concentration of 10 mg/ml in DMSO) were dissolved in a sufficient amount of anhydrous DMSO to give a final volume of 1 ml. The reaction was stirred at room temperature for 1 hour, after which 90 mg of CPG 2000 A silica beads (Control Pores Glass) or 100 mg of glass beads or 100 mg of polystyrene beads functionalized with an amino spacer were added to the solution. The reaction was continued overnight at room temperature. The supports were then filtered off and washed thoroughly with DMSO and with anhydrous acetone, and then dried under vacuum in the presence of calcium chloride.

These supports were then used to carry out oligonucleotide syntheses as described in Example 1. If the monomer (II), a peptide synthesis primer, is coupled to the polymer, peptide syntheses can be developed on the supports, using Fmoc chemistry

### EXAMPLE 7

Same protocol for synthesizing the spherical supports as in Example 1 or Example 6, using starting monomers for oligonucleotide syntheses of type (I), or using starting monomers for peptide syntheses of type (II) or using starting monomers of type (III) or (IV) comprising a photolabile protecting group on the site of initiation of synthesis of the biopolymer.

The photolabile group used can be, for example, 6-nitroveratryl, 6-nitropiperonyl, methyl-6-nitroveratryl, nitroveratryloxycarbonyl, methyl-6-nitropiperonyl, nitrobenzyl, nitrobenzyloxycarbonyl, dimethyldimethoxybenzyl, dimethyldimethoxybenzyloxy-carbonyl, 5-bromo-7-nitroindolinyl, hydroxymethyl-cinnamoyl, 2-oxymethylene anthraquinone, pyrenyl-methoxycarbonyl. The biopolymer synthesis will be primed by exposing the support to a suitable range of wavelengths.

### EXAMPLE 8

Same concept as in Example 7, using flat supports (silicon wafer, silica microplate) silanized at the surface with an aminosilane and then coated with a linear polymer based on maleic anhydride. The amino functions of the spacer arm react with the hyrophilic functions of the polymer to create an amide function which is stable in basic medium. By grafting the monomer (I) and/or (II) and/or (III) and/or (IV) onto the polymer, oligonucleotide and/or peptide syntheses can be primed in a controlled manner. The plates are pretreated in acidic or basic medium, or alternatively, if a system of specific masking groups is used, they can be exposed to radiation on delimited regions of their surface, in order to remove the photolabile groups. With the reactive functions thus freed, biopolymer syntheses can be developed at the surface by the conventional methods of chemistry on a support. These functionalized matrices can be used for sequencing genes or screening antibodies.

1. Complex chemical compound comprising:
a solid support,
surface chemical groups linked to the solid support via covalent bonding,
a conjugate comprising an organic polymer comprising chemical side functions linked to the surface groups, and chemical side residues
characterized in that starting biomonomers, which are optionally chemically modified, are linked covalently, on the one hand to the chemical residues of said organic polymer, respectively, and on the other hand each to a biopolymer.

2. Compound according to claim 1, characterized in that the covalent bonding of the starting biomonomers to the chemical residues of the organic polymer cannot be cleaved under the conditions for cleaving the support/organic polymer bond.

3. Compound according to claim 1, characterized in that the side functions of the reactive organic polymer are of electrophilic and/or thiol and/or disulfide bridge type.

4. Chemical compound according to claim 1, characterized in that the starting biomonomers, which may be identical or different, are nucleosides and/or nucleotides and/or amino acids and/or saccharides, which may be natural or modified.

5. Compound according to claim 4, characterized in that the starting biomonomers are both nucleotides and amino acids.

6. Compound according to claim 1, characterized in that the starting biomonomers are extended and polymerized with other synthons, each according to a predetermined sequence of said synthons, in order to obtain mixed biopolymers, such as oligonucleotides and/or peptides linked to the organic polymer.

7. Chemical compound according to claim 6, characterized in that the mixed biopolymers are of oligonucleotide and peptide or oligonucleotide-peptide type.

8. Complex chemical compound according to claim 7, characterized in that the biopolymers fort, with the organic polymer to which they are linked, ligands which can be linked directly or indirectly to anti-ligands.

9. Process for the chemical synthesis of a compound according to claim 1, comprising the following steps:
a) a functional solid support comprising surface groups is provided;
b) at least one reactive organic polymer is provided, the backbone of which comprises, on the one hand, chemical side functions complementary to the surface groups of the solid support, and, on the other hand, chemical side residues;
c) starting biomonomers, which may be identical or different, for biopolymerization are provided, comprising, on the one hand, a reactive substituent, and, on the other hand, a protective group;
d) at least said organic polymer is reacted:
either with the solid support, to establish a covalent bond between the surface groups of said solid support and the side functions of said organic polymer, after which the organic polymer linked to the solid support is reacted with the starting biomonomers, to graft these biomonomers covalently, directly Or indirectly, with the side residues of said organic polymer;
or with the starting biomonomers, to graft these biomonomers covalently, directly or indirectly, with the side residues of said organic polymer, after which the organic polymer linked to the starting biomonomers is reacted with the solid support, to establish a covalent bond between the surface groups of said solid support and the side functions of said organic polymer.

10. Process according to claim 9, characterized in that it comprises, after step c and before step d, the following step:
c′) the organic polymer is reacted with a reagent which generates spacer arms, to graft spacer arms onto the side functions of said organic polymer, respectively, each spacer arm comprising a reactive function at its free end.

11. Complex chemical compound comprising:
a solid support,
surface chemical groups linked to the solid support via covalent bonding,
at least one conjugate comprising an organic polymer comprising chemical side functions linked to the surface groups, and chemical side residues,
characterized in that starting biomonomers, i.e. chemically modified biomonomers, are linked covalently to the chemical residues of said organic polymer, respectively.

12. Use of the compound according to claim 11, to carry out the synthesis of a ligand, comprising the steps in which:
f) identical or different synthons are provided,
g) the biopolymer chains are grown from starting biomonomers of the compound, respectively, by successive cycles of coupling/deprotection, according to at least one same predetermined sequence of synthons,
h) the support/organic polymer bond is cleaved.

13. Use according to claim 12, comprising, between steps g and h, the step in which:
i) biopolymer chains are grown from other starting biomonomers, respectively, by successive cycles of coupling/deprotection of the synthons, according to at least one same other predetermined sequence of synthons.

14. Use according to claim 12, characterized in that step (h) is carried out by chemical or enzymatic or thermal or photochemical reaction.

15. Reagent, characterized in that it comprises a chemical compound according to any one of claims 1 to 11.
